(19) United States
(12) Patent Application Publication
(10) Pub. No.: US 2012/0258878 A1
(43) Pub. Date: Oct. 11, 2012

(54) PROGNOSTIC GENE SIGNATURES FOR NON-SMALL CELL LUNG CANCER

(75) Inventor: Fadia Saad, Victoria (CA)

(73) Assignee: Med Biogene Inc., Vancouver (CA)

(21) Appl. No.: 13/198,228

(22) Filed: Aug. 4, 2011

Related U.S. Application Data
(60) Provisional application No. 61/370,690, filed on Aug. 4, 2010.

(51) Int. Cl. C40B 30/04; C40B 40/06; G06F 19/24; C12Q 1/68

(52) U.S. Cl. 506/9; 435/6.12; 435/6.11; 506/16; 702/19

(57) ABSTRACT

The application provides methods of prognosing and classifying lung cancer patients into poor survival groups or good survival groups by way of a multigene signature, comprising at least 5 genes from Table 3. The application also includes kits and computer products for use in the methods of the application.

This application claims priority to and the benefit of U.S. Provisional Application No. 61/370,690, filed Aug. 4, 2010, which is hereby incorporated by reference in its entirety.

### FIELD

The application relates to compositions and methods for prognosing and classifying non-small cell lung cancer.

### BACKGROUND OF THE INVENTION

In North America, lung cancer is the leading cancer in males and the leading cause of cancer deaths in both males and females¹. Non-small cell lung cancer (NSCLC) represents 80% of all lung cancers and has an overall 5-year survival rate of only 16%¹. Tumor stage is the primary determinant for treatment selection for NSCLC patients. Recent clinical trials have led to the adoption of adjuvant cisplatin-based chemotherapy in early stage NSCLC patients (Stages IB-IIIA). The 5-year survival advantage conferred by adjuvant chemotherapy in recent trials are 4% in the International Adjuvant Lung Trial (IALT) involving 1,867 stage I-III patients², 15% in the National Cancer Institute of Canada Clinical Trials Group (NCIC CTG) BR.10 Trial involving 482 stage IB-II patients³, and 9% in the Adjuvant Navelbine International Trialist Association (ANITA) trial involving 840 stage IB-IIIA patients. Pre-planned stratification analysis in the later two trials showed no significant survival benefit for stage IB patients^{3,4}. This was also demonstrated in the Cancer and Leukemia Group (CALGB) Trial 9633 that tested the benefit of chemotherapy on 344 stage 13 patients receiving carboplatin and paclitaxel or observation⁵. Although initially presented in 2004 as a positive trial, recent survival analyses show no significant survival advantage with chemotherapy for either disease-free survival (HR=0.80, p=0.065) or overall survival (HR=0.83, p=0.12)⁵. In an attempt to draw an overall conclusion regarding the effectiveness of adjuvant cisplatin-based chemotherapy, the Lung Adjuvant Cisplatin Evaluation (LACE) meta-analysis⁶ was conducted which synthesized information from the 5 largest published, cisplatin-based trials that did not administer concurrent thoracic radiation [Adjuvant Lung Project Italy (ALPI)⁷, Big Lung Trial (BLT)⁸, IALT², BR.10³, and ANITA⁹]. The study found a 5.3% absolute survival advantage at 5-year (HR-0.89, 95% CI 0.82-0.96, p=0.004). However, stratified analysis by stage showed that the stage IB patients did not benefit significantly from cisplatin treatment (HR=0.92, 95% CI 0.78-1.10). Moreover, a detriment for chemotherapy was suggested in stage IA patients (HR=1.41, 95% CI 0.96-2.09)⁶. Therefore, the current standard of treatment for patients with stage I NSCLC remains surgical resection alone. However, 30 to 40 percent of these stage I patients are expected to relapse after the initial surgery^{10,11}, indicating that a subgroup of these patients might benefit from adjuvant chemotherapy.

The lack of consistent prognostic molecular markers for early stage NSCLC patients led to attempts to identify novel gene expression signatures using genome wide microarray platforms. Such multi-gene signatures might be stronger than individual genes to predict poor prognosis and poor prognostic patients could potentially benefit from adjuvant therapies. Previous microarray studies have identified prognostic signatures that demonstrated minimal overlaps in the gene sets.¹²⁻²⁰. While only one of the early studies involved secondary signature validation in independent datasets¹², all recently reported signatures were tested for validation^{13-16,20}. Nevertheless, lack of direct overlaps between signatures remains. One of the potential confounding factors is that signatures were derived from patients operated at single institutions, which may introduce biases.

### SUMMARY OF THE INVENTION

As discussed in the Background section, certain patients suffering from NSCLC benefit from adjuvant chemotherapy. Attempts to identify systematically patient subpopulations in which adjuvant therapy would lead to increased survival or improve patient prognosis have generally failed. Efforts to assemble prognostic molecular markers have yielded various non-overlapping gene sets but have fallen short of establishing a gene signature independent of other clinical factors (eg. histology, age) that serves as a reliable classifier for prognosis.

As will be discussed in more detail below, Applicants have identified from historical patient data a set of forty genes whose expression levels can be used in a gene signature that is prognostic of survival outcome. The forty genes are provided in Table 3. The prognostic value of the 40 genes identified by Applicants was verified by validation against independent data sets, as set forth in the Examples below. The present disclosure provides methods and kits useful for obtaining and utilizing expression information for the forty genes, and subsets thereof, to obtain prognostic information for patients with NSCLC.

The methods of the present disclosure are useful in prognosing or classifying a subject with NSCLC into a poor survival group or a good survival group by determining relative expression levels of a set of genes described herein, and in some embodiments combining the expression levels with gene-specific coefficients, or reference values, to generate a score for the subject. This score, referred to as a risk score, is compared to a control value and permits the subject to be classified as belonging to a poor survival group or a good survival group depending on whether the risk score is greater or less than the control value.

The methods of the present disclosure involve obtaining from a patient tumor specimen relative expression data (e.g., a gene expression profile), at the DNA, mRNA, miRNA, or protein level, for a set of genes comprising at least 5, at least 10, at least 15, at least 25, at least 30, or at least 35 genes listed in Table 3, or comprising the 40 genes listed in Table 3. In some embodiments, the set of genes or the gene expression profile contains the expression levels for less than 2000 genes in total, or in other embodiments less than 1000 genes, less than 500 genes, less than 100 genes, or less than 50 genes, while including the genes listed in Table 3 (or subset thereof). Such a gene expression profile is indicative of survival and/or outcome for NSCLC, and may be indicative of whether the patient will benefit from chemotherapy. In various embodiments, this data is processed to determine a score or test value, and the score or test value is compared to one or more reference values. Relative expression levels are expression data normalized according to techniques known to those skilled in the art. Expression data may be normalized with respect to one or more genes with invariant expression, such as “housekeeping” genes. In some embodiments, expression data may be processed using standard techniques, such as transformation to a z-score, and/or software tools, such as RMAexpress v0.3.

In some embodiments, the risk score can be generated by calculating the sum over each of the genes in Table 3, or subset thereof as described, of: the inner product of reference values reported in Table 3 and the relative expression level for the corresponding gene in a sample.

Control values are established from historical expression data for each of the genes in the multi-gene signature. In some embodiments, the control value used in the method is selected based on the subject's disease stage. For example, where a subject has Stage IA NSCLC, a control value of 0.15 is used in prognosing the subject. Where a subject has Stage IB NSCLC, a control value of 0.00 is used in prognosing the subject. Where a subject has Stage II NSCLC, a control value of −0.05 is used in prognosing the subject.

Accordingly, in one embodiment, the application provides a method of prognosing or classifying a subject with non-small cell lung cancer comprising the steps:
a. determining the relative expression of at least 5, at least 10, at least 15, at least 25, at least 30, at least 35, or at least 40 biomarkers in a test sample from the subject, wherein the biomarkers correspond to genes in Table 3,
b. multiplying the relative expression of each of the biomarkers by a reference value for the corresponding biomarker,
c. calculating a risk score for the test sample by summing the values obtained in step (b), and
d. comparing the risk score calculated for the test sample with a control value,
   wherein a risk score above said control value is used to prognose or classify the subject with NSCLC into a good survival group and a risk score below said control value is used to prognose or classify the subject with NSCLC into a poor survival group.

In some embodiments, a method is provided whereby a subject with NSCLC is prognosed comprising the steps of:
(a) determining relative expression levels of at least 5, at least 10, at least 15, at least 25, at least 30, at least 35, or at least 40 biomarkers from Table 3,
(b) calculating a risk score for the subject from the expression levels, and,
(c) comparing the risk score to a control value,
   Wherein a risk score greater than the control value is used to classify a subject into a high risk or poor survival group and a risk score lower than the control value is used to classify a subject into a lower risk or good survival group.

Another aspect of the application provides compositions for use with the methods described herein.

The application also provides for kits used to prognose or classify a subject with NSCLC into a good survival group or a poor survival group or for selecting therapy for a subject with NSCLC that includes detection agents that can detect the expression products of the biomarkers.

In one aspect, the present disclosure provides kits useful for carrying out the prognostic tests described herein. The kits generally comprise reagents and compositions for obtaining relative expression data for the forty genes described in Table 3, or subsets thereof described herein, including subsets of at least 5, at least 10, at least 15, at least 25, at least 30, at least 35 genes listed in Table 3, or the 40 genes listed in Table 3. In some embodiments, the kit comprises reagents and compositions for obtaining relative expression data for less than 2000 genes in total, or in other embodiments less than 1000 genes, less than 500 genes, less than 100 genes, or less than 50 genes, while including the genes listed in Table 3 (or subset thereof). As will be recognized by the skilled artisans, the contents of the kits will depend upon the means used to obtain the relative expression information.

Kits may comprise a labeled compound or agent capable of detecting protein product(s) or nucleic acid sequence(s) in a sample and means for determining the amount of the protein, mRNA, or miRNA in the sample (e.g., an antibody which binds the protein or a fragment thereof, or an oligonucleotide probe which binds to DNA or mRNA encoding the protein). Kits can also include instructions for interpreting the results obtained using the kit.

In some embodiments, the kits are oligonucleotide-based kits, which may comprise, for example: (1) an oligonucleotide, e.g., a detectably labeled oligonucleotide, which hybridizes to a nucleic acid sequence encoding a marker protein or (2) a pair of primers useful for amplifying a marker nucleic acid molecule. Kits may also comprise, e.g., a buffering agent, a preservative, or a protein stabilizing agent. The kits can further comprise components necessary for detecting the detectable label (e.g., an enzyme or a substrate). The kits can also contain a control sample or a series of control samples which can be assayed and compared to the test sample. Each component of a kit can be enclosed within an individual container and all of the various containers can be within a single package, along with instructions for interpreting the results of the assays performed using the kit.

In some embodiments, the kits are antibody-based kits, which may comprise, for example: (1) a first antibody (e.g., attached to a solid support) which binds to a marker protein; and, optionally, (2) a second, different antibody which binds to either the protein or the first antibody and is conjugated to a detectable label.

A further aspect provides computer implemented products, computer readable mediums and computer systems that are useful for the methods described herein.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIGS. 1A-D provides plots of the probability of an event by site (1A), cohort (1B), histology (1C), and cancer stage (1D) for the patient datasets used to develop the prognostic signature.

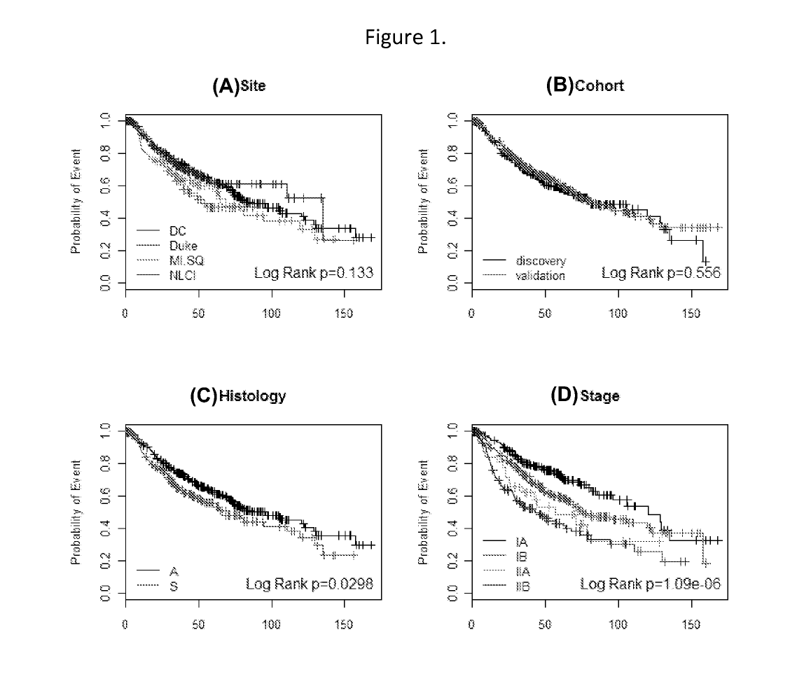

FIG. 2 provides a flow chart of the protocol for derivation and testing of the prognostic signature.

FIG. 3 shows graphs of cross validation using the Concordance index (C-index) as an indicator of performance for two different methodologies (NTP and Lasso). Solid lines indicate median performance, dotted lines represent the 25th and 75th percentiles.

FIG. 4 shows three graphs of the probability of an event as a measure of the performance of the 40-gene signature in a validation test for clinical data across all stages of disease (FIG. 4A) and broken out by stage (FIG. 4B for Stage IB and FIG. 4C for Stage II).

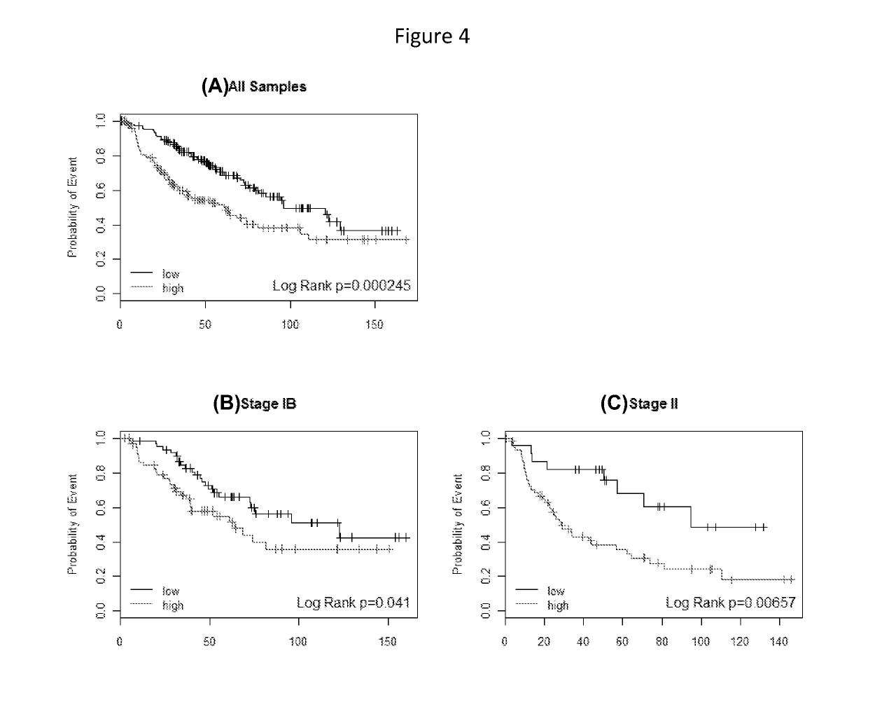

FIG. 5 shows a graph of the Concordance (C) index for datasets based on clinical data alone (top bar), 40-gene signature alone (middle bar), or a combination of both (bottom bar).

### DETAILED DESCRIPTION OF THE INVENTION

The application relates to 40 biomarkers, and various subsets thereof, that form gene signatures, and provides methods, compositions, computer implemented products, detection agents and kits for prognosing or classifying a subject with non-small cell lung cancer (NSCLC). Using available gene expression datasets compiled from subjects diagnosed with NSCLC, Applicants have developed gene signatures that are prognostic of disease outcome in subjects with resectable lung cancer. For example, a multi-gene signature was developed through modeling of individual genes using nearest template prediction (NTP), calculating the CoxPH statistic for all genes, ranking genes by the absolute value of the statistic and selecting the top N genes. Test cases were then scored using the sum, over all genes in the signature, of the inner product of the vector of CoxPH statistics and the relative expression level for each biomarker in the test sample.

In an aspect, a multi-gene signature comprising at least 40 genes is prognostic of clinical outcome. The signature comprises the identity of each gene, or biomarker, in the signature and one or more gene-specific coefficients for each biomarker. The biomarkers in the multi-gene signature include at least the 40 genes listed in Table 3, and optional additional genes. In one embodiment, the signature is a 40-gene signature comprising the 40 genes listed in Table 3 and a single reference value for each of the biomarkers in the signature. Table 3 provides an example of reference values for each of the 40 biomarkers listed.

In certain embodiments, the multi-gene signature is based on a subset of the 40 genes, including at least 5, at least 10, at least 15, at least 25, at least 30 genes, or at least 35 genes listed in Table 3, where such signature is indicative of outcome or survival of a subject with NSCLC.

In some embodiments, the gene signature is used in prognosing or classifying a subject in the early stages of NSCLC. Accordingly, in one embodiment, the subject has stage I NSCLC, for example, Stage IA or Stage IB. In another embodiment, the subject has stage II NSCLC.

As disclosed herein, relative expression data (e.g., a gene expression profile) from a patient can be combined with reference values on a gene-by-gene basis for each of forty genes, or subset thereof as described, to generate a test value which allows prognosis. In some embodiments, relative expression data are subjected to an algorithm that yields a single test value, or risk score, which is then compared to a control value obtained from the historical expression data for a patient or pool of patients.

In some embodiments, the control value is a numerical threshold for predicting outcomes, for example good and poor outcome. In some embodiments, a test value or risk score greater than the control value is predictive, for example, of a poor outcome, whereas a risk score falling below the control value is predictive, for example, of a good outcome.

In some embodiments, a method for prognosing or classifying a subject with NSCLC comprises:
(a) determining relative expression levels of at least 40 biomarkers from Table 3, or subset thereof,

(b) calculating a risk score for the subject from the expression levels, and,
(c) comparing the risk score to a control value,
   Wherein a risk score greater than the control value is used to classify a subject into a high risk or poor survival group and a risk score lower than the control value is used to classify a subject into a lower risk or good survival group.

In some embodiments, the risk score for a test sample is the sum for all of the genes in the multi-gene signature of: the inner product of a gene-specific reference value and the relative expression level of the corresponding gene in the test sample.

Relative expression levels are expression data normalized according to techniques known to those skilled in the art. Expression data may be normalized with respect to one or more genes with invariant expression, such as “housekeeping” genes, as described below. In some embodiments, expression data may be processed using standard techniques, such as transformation to a z-score, and/or software tools, such as RMAexpress v0.3.

The term “biomarker” as used herein refers to a gene that is differentially expressed in individuals with non-small cell lung cancer (NSCLC) according to prognosis and is predictive of different survival outcomes. In some embodiments, a 40-gene signature comprises 40 biomarkers listed in Table 3. In other embodiments, the biomarkers comprise the expression levels of a subset of the of the genes listed in Table 3, such as at least 5, at least 10, at least 15, at least 25, or at least 30 genes, or at least 35 genes listed in Table 3

The term “reference expression profile” as used herein refers to the expression of the 40 biomarkers or genes listed in Table 3, or subset thereof, and which are associated with a clinical outcome in a NSCLC patient. The reference expression profile comprises at least one value representing the expression level of each biomarker, wherein each biomarker corresponds to one gene in Table 3. The reference expression profile is identified using one or more samples comprising tumor wherein the expression is similar between related samples defining an outcome class or group such as poor survival or good survival and is different to unrelated samples defining a different outcome class such that the reference expression profile is associated with a particular clinical outcome. The reference expression profile is accordingly a reference profile of the expression of the genes in Table 3 (or subset thereof), to which the subject expression levels of the corresponding genes in a patient sample are compared in methods for determining or predicting clinical outcome.

As used herein, the term “control value” refers to a specific value can be used to prognose or classify a subject into an outcome class. Expression data of the biomarkers in the dataset can be used to create a “control value” that is used in evaluating samples from test subjects. A control value is obtained from the historical expression data for a patient or pool of patients with a known outcome. In some embodiments, the control value is a numerical threshold for predicting outcomes, for example good and poor outcome.

In some embodiments, the “control” is a predetermined value for the set of biomarkers obtained from NSCLC patients whose biomarker expression values and survival times are known. Using values from known samples allows one to develop an algorithm for classifying new patient samples into good and poor survival groups. Such an algorithm is described in the Example.

As used herein, a “reference value” refers to a gene-specific coefficient derived from historical expression data. The multi-gene signatures of the present disclosure comprise reference values for each gene in the signature. In some embodiments, the multi-gene signature comprises one reference value for each gene in the signature. In some embodiments, the multi-gene signature is a 40-gene signature and comprises forty reference values, one for each gene in the signature.

The term “differentially expressed” or “differential expression” as used herein refers to a difference in the level of expression of the biomarkers that can be assayed by measuring the level of expression of the products of the biomarkers, such as the difference in level of messenger RNA transcript expressed or proteins expressed of the biomarkers. In a preferred embodiment, the difference is statistically significant. The term “difference in the level of expression” refers to an increase or decrease in the measurable expression level of a given biomarker as measured by the amount of messenger RNA transcript and/or the amount of protein in a sample as compared with the measurable expression level of a given biomarker in a control. In one embodiment, the differential expression can be compared using the ratio of the level of expression of a given biomarker or biomarkers as compared with the expression level of the given biomarker or biomarkers of a control, wherein the ratio is not equal to 1.0. For example, an RNA or protein is differentially expressed if the ratio of the level of expression in a first sample as compared with a second sample is greater than or less than 1.0. For example, a ratio of greater than 1, 1.2, 1.5, 1.7, 2, 3, 3, 5, 10, 15, 20 or more, or a ratio less than 1, 0.8, 0.6, 0.4, 0.2, 0.1, 0.05, 0.001 or less. In another embodiment the differential expression is measured using p-value. For instance, when using p-value, a biomarker is identified as being differentially expressed as between a first sample and a second sample when the p-value is less than 0.1, preferably less than 0.05, more preferably less than 0.01, even more preferably less than 0.005, the most preferably less than 0.001.

The term “similarity in expression” as used herein means that there is no or little difference in the level of expression of the biomarkers between the test sample and the control or reference profile. For example, similarity can refer to a fold difference compared to a control. In a preferred embodiment, there is no statistically significant difference in the level of expression of the biomarkers.

The term “most similar” in the context of a reference profile refers to a reference prone that is associated with a clinical outcome that shows the greatest number of identities and/or degree of changes with the subject profile.

The term “prognosis” as used herein refers to a clinical outcome such as a poor survival or a good survival associated with a disease subtype. The prognosis provides an indication of disease progression and includes an indication of likelihood of death due to lung cancer. In one embodiment the clinical outcome classes include a good survival group and a poor survival group.

The terms “prognosing” and “classifying” as used herein mean categorizing a subject into a clinical outcome group, such as a poor survival group or a good survival group. In some embodiments, a subject is classified or prognosed according to whether the subjects risk score is above or below a control value. For example, prognosing or classifying comprises a method or process of determining whether an individual with NSCLC has a good or poor survival outcome, or grouping an individual with NSCLC into a good survival group or a poor survival group, based on whether the individual's calculated risk score is above or below the control value.

The term “good survival” as used herein refers to an increased chance of survival as compared to patients in the “poor survival” group. For example, the biomarkers of the application can prognose or classify patients into a “good survival group”. These patients are at a lower risk of death after surgery. In some embodiments, the patient is classified in a good survival group, and the patient does not receive chemotherapy.

The term “poor survival” as used herein refers to an increased risk of death as compared to patients in the “good survival” group. For example, gene signatures of the application can prognose or classify patients into a “poor survival group”. These patients are at greater risk of death after surgery. In some embodiments, the patient is classified in a poor survival group, and the patient receives a chemotherapeutic regimen.

The term “subject” as used herein refers to any member of the animal kingdom that may be inflicted with NSCLC, preferably a human being who has NSCLC or is suspected of having NSCLC.

NSCLC patients are classified into stages, which are used to determine therapy. For example, stage I includes cancer in the lung, but has not spread to adjacent lymph nodes or outside the chest. Stage I is divided into two categories based on the size of the tumor (IA and IB). Stage II includes cancer located in the lung and proximal lymph nodes. Stage II is divided into 2 categories based on the size of tumor and nodal status (IIA and IIB), Stage III includes cancer located in the lung and the lymph nodes. Stage III is divided into 2 categories based on the size of tumor and nodal status (IIIA and IIIB). Suitable subjects are those whose tumors are resectable or treatable by surgery. Typically, suitable subjects have early stage NSCLC. The term “early stage NSCLC” includes patients with Stage I to IIIA NSCLC. These patients are treated primarily by complete surgical resection. Staging is done based on a series of tests. Testing may include any or all of the following: history, physical examination, routine laboratory evaluations, chest x-rays, and chest computed tomography scans or positron emission tomography scans with infusion of contrast materials.

Thus, a classification algorithm or “class predictor” may be constructed to classify samples. The process for preparing a suitable class predictor is reviewed in R. Simon, Diagnostic and prognostic prediction using gene expression profiles in high-dimensional microarray data, British Journal of Cancer (2003) 89, 1599-1604, which review is hereby incorporated by reference in its entirety.

The term “test sample” as used herein refers to any cancer-affected fluid, cell or tissue sample from a subject which can be assayed for biomarker expression products and/or a reference expression profile, e.g. genes differentially expressed in subjects with NSCLC according to survival outcome. In certain embodiments, the test sample is a frozen tissue specimen or is a formalin-fixed paraffin-embedded tumor tissue sample, or is a cultured tumor specimen.

The phrase “determining the expression of biomarkers” as used herein refers to determining or quantifying RNA or proteins expressed by the biomarkers. The term “RNA” includes microRNA (or “miRNA”), mRNA transcripts, and/or specific spliced variants of mRNA. The terms “RNA product of the biomarker,” “biomarker RNA,” or “target RNA” as used herein refers to RNA transcripts transcribed from the biomarkers and/or specific spliced variants. In the case of “protein”, it refers to proteins translated from the RNA transcripts transcribed from the biomarkers. The term “protein product of the biomarker” or “biomarker protein” refers to proteins translated from RNA products of the biomarkers.

A person skilled in the art will appreciate that a number of methods can be used to detect or quantify the level of RNA products of the biomarkers within a sample, including arrays, such as microarrays, RT-PCR (including quantitative PCR), nuclease protection assays and Northern blot analyses. Any analytical procedure capable of permitting specific and quantifiable (or semi-quantifiable) detection of the biomarkers may be used in the methods herein presented, such as the microarray methods set forth herein, and methods known to those skilled in the art.

Accordingly, in one embodiment, the biomarker expression levels are determined using arrays, optionally microarrays, RT-PCR, optionally quantitative RT-PCR, nuclease protection assays or Northern blot analyses.

In some embodiments, the biomarker expression levels are determined by using an array. cDNA microarrays consist of multiple (usually thousands) of different cDNAs spotted (usually using a robotic spotting device) onto known locations on a solid support, such as a glass microscope slide. Microarrays for use in the methods described herein comprise a solid substrate onto which the probes are covalently or non-covalently attached. The cDNAs are typically obtained by PCR amplification of plasmid library inserts using primers complementary to the vector backbone portion of the plasmid or to the gene itself for genes where sequence is known. PCR products suitable for production of microarrays are typically between 0.5 and 2.5 kB in length. In a typical microarray experiment, RNA (either total RNA or poly A RNA) is isolated from cells or tissues of interest and is reverse transcribed to yield cDNA. Labeling is usually performed during reverse transcription by incorporating a labeled nucleotide in the reaction mixture. A microarray is then hybridized with labeled RNA, and relative expression levels calculated based on the relative concentrations of cDNA molecules that hybridized to the cDNAs represented on the microarray. Microarray analysis can be performed by commercially available equipment, following manufactuer's protocols, such as by using Affymetrix GeneChip technology, Agilent Technologies microarrays, lumina Whole-Genome DASL array assays, or any other comparable microarray technology.

In some embodiments, probes capable of hybridizing to one or more biomarker RNAs or cDNAs are attached to the substrate at a defined location (“addressable array”). Probes can be attached to the substrate in a wide variety of ways, as will be appreciated by those in the art. In some embodiments, the probes are synthesized first and subsequently attached to the substrate. In other embodiments, the probes are synthesized on the substrate. In some embodiments, probes are synthesized on the substrate surface using techniques such as photopolymerization and photolithography.

In some embodiments, microarrays are utilized in a RNA-primed, Array-based Klenow Enzyme (“RAKE”) assay. See Nelson, P. T. et al. (2004) Nature Methods 1(2):1-7; Nelson, P. T. et al. (2006) RNA 12(2):1-5, each of which is incorporated herein by reference in its entirety. In these embodiments, total RNA is isolated from a sample. Optionally, small RNAs can be further purified from the total RNA sample. The RNA sample is then hybridized to DNA probes immobilized at the 5-end on an addressable array. The DNA probes comprise a base sequence that is complementary to a target RNA of interest, such as one or more biomarker RNAs capable of specifically hybridizing to a nucleic acid comprising a sequence that is identically present in one of the genes listed in Table 3 under standard hybridization conditions.

In some embodiments, the addressable array comprises DNA probes for no more than 2000 genes, or no more than 1000 genes, or no more than 500 genes, or no more than 200 genes, or no more than 100 genes, while including the set of genes from Table 3, or a subset thereof as described herein. In some embodiments, the addressable array comprises or consists essentially of DNA probes for the 40 genes listed in Table 3. In this context, the term “consists essentially of” means that the array may contain other genes for normalizing signals or expression levels, but which do not directly contribute to the score or classification.

In some embodiments, quantitation of biomarker RNA expression levels requires assumptions to be made about the total RNA per cell and the extent of sample loss during sample preparation. In some embodiments, the addressable array comprises DNA probes for each of the 40 genes listed in Table 3 (or subset thereof) and, optionally, one, two, three, or four housekeeping genes.

In some embodiments, expression data are pre-processed to correct for variations in sample preparation or other non-experimental variables affecting expression measurements. For example, background adjustment, quantile adjustment, and summarization may be performed on microarray data, using standard software programs such as RMAexpress v0.3, followed by centering of the data to the mean and scaling to the standard deviation.

After the sample is hybridized to the array, it is exposed to exonuclease I to digest any unhybridized probes. The Klenow fragment of DNA polymerase I is then applied along with biotinylated dATP, allowing the hybridized biomarker RNAs to act as primers for the enzyme with the DNA probe as template. The slide is then washed and a streptavidin-conjugated fluorophore is applied to detect and quantitate the spots on the array containing hybridized and Klenow-extended biomarker RNAs from the sample.

In some embodiments, the RNA sample is reverse transcribed using a biotin/poly-dA random octamer primer. The RNA template is digested and the biotin-containing cDNA is hybridized to an addressable microarray with bound probes that permit specific detection of biomarker RNAs. In typical embodiments, the microarray includes at least one probe comprising at least 8, at least 9, at least 10, at least 11, at least 12, at least 13, at least 14, at least 15, at least 16, at least 17, at least 18, at least 19, even at least 20, 21, 22, 23, or 24 contiguous nucleotides identically present in each of the genes listed in Table 3. After hybridization of the cDNA to the microarray, the microarray is exposed to a streptavidin-bound detectable marker, such as a fluorescent dye, and the bound cDNA is detected. See Liu C. G. et al. (2008) Methods 44:22-30, which is incorporated herein by reference in its entirety.

In one embodiment, the array is a U133A chip from Affymetrix. In another embodiment, a plurality of nucleic acid probes that are complementary or hybridizable to an expression product of the genes listed in Table 3 are used on the array.

The term “nucleic acid” includes DNA and RNA and can be either double stranded or single stranded.

The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed.

The term “probe” as used herein refers to a nucleic acid sequence that will hybridize to a nucleic acid target sequence. In one example, the probe hybridizes to an RNA product of the biomarker or a nucleic acid sequence complementary thereof. The length of probe depends on the hybridization conditions and the sequences of the probe and nucleic acid target sequence. In one embodiment, the probe is at least 8, 10, 15, 20, 25, 50, 75, 100, 150, 200, 250, 400, 500 or more nucleotides in length.

In some embodiments, compositions are provided that comprise at least one biomarker or target RNA-specific probe. The term “target RNA-specific probe” encompasses probes that have a region of contiguous nucleotides having a sequence that is either (i) identically present in one of the genes listed in Tables 3 or 4, or (ii) complementary to the sequence of a region of contiguous nucleotides found in one of the genes listed in Table 3, where “region” can comprise the full length sequence of any one of the genes listed in Table 3, a complementary sequence of the full length sequence of any one of the genes listed in Table 3, or a subsequence thereof.

In some embodiments, target RNA-specific probes consist of deoxyribonucleotides. In other embodiments, target RNA-specific probes consist of both deoxyribonucleotides and nucleotide analogs. In some embodiments, biomarker RNA-specific probes comprise at least one nucleotide analog which increases the hybridization binding energy. In some embodiments, a target RNA-specific probe in the compositions described herein binds to one biomarker RNA in the sample.

In some embodiments, more than one probe specific for a single biomarker RNA is present in the compositions, the probes capable of binding to overlapping or spatially separated regions of the biomarker RNA.

It will be understood that in some embodiments in which the compositions described herein are designed to hybridize to cDNAs reverse transcribed from biomarker RNAs, the composition comprises at least one target RNA-specific probe comprising a sequence that is identically present in a biomarker RNA (or a subsequence thereof).

In some embodiments, a biomarker RNA is capable of specifically hybridizing to at least one probe comprising a base sequence that is identically present in one of the genes listed in Table 3. In some embodiments, a biomarker RNA is capable of specifically hybridizing to at least one probe comprising a base sequence that is identically present in one of the genes listed in Table 3.

In some embodiments, the composition comprises a plurality of target or biomarker RNA-specific probes each comprising a region of contiguous nucleotides comprising a base sequence that is identically present in one or more of the genes listed in Table 3, or in a subsequence thereof.

As used herein, the terms “complementary” or “partially complementary” to a biomarker or target RNA (or target region thereof), and the percentage of “complementarity” of the probe sequence to that of the biomarker RNA sequence is the percentage “identity” to the reverse complement of the sequence of the biomarker RNA. In determining the degree of “complementarily” between probes used in the compositions described herein (or regions thereof) and a biomarker RNA, such as those disclosed herein, the degree of “complementarity” is expressed as the percentage identity between the sequence of the probe (or region thereof) and the reverse complement of the sequence of the biomarker RNA that best aligns therewith. The percentage is calculated by counting the number of aligned bases that are identical as between the 2 sequences, dividing by the total number of contiguous nucleotides in the probe, and multiplying by 100.

In some embodiments, the microarray comprises probes comprising a region with a base sequence that is fully complementary to a target region of a biomarker RNA. In other embodiments, the microarray comprises probes comprising a region with a base sequence that comprises one or more base mismatches when compared to the sequence of the best-aligned target region of a biomarker RNA.

As noted above, a “region” of a probe or biomarker RNA, as used herein, may comprise or consist of 8, 9, 10, 11, 12, 13, 14, 15, 16, 17, 18, 19, 20, 21, 22, 23, 24, 25, 26, 27, 28, 29 or more contiguous nucleotides from a particular gene or a complementary sequence thereof. In some embodiments, the region is of the same length as the probe or the biomarker RNA. In other embodiments, the region is shorter than the length of the probe or the biomarker RNA.

In some embodiments, the microarray comprises forty probes each comprising a region of at least 10 contiguous nucleotides, such as at least 11 contiguous nucleotides, such as at least 13 contiguous nucleotides, such as at least 14 contiguous nucleotides, such as at least 15 contiguous nucleotides, such as at least 16 contiguous nucleotides, such as at least 17 contiguous nucleotides, such as at least 18 contiguous nucleotides, such as at least 19 contiguous nucleotides, such as at least 20 contiguous nucleotides, such as at least 21 contiguous nucleotides, such as at least 22 contiguous nucleotides, such as at least 23 contiguous nucleotides, such as at least 24 contiguous nucleotides, such as at least 25 contiguous nucleotides with a base sequence that is identically present in one of the genes listed in Table 3.

In some embodiments, the microarray component comprises probes each comprising a region with a base sequence that is identically present in each of the genes listed in Table 3, or subset thereof.

In another embodiment, the biomarker expression levels are determined by using quantitative RT-PCR. RT-PCR is one of the most sensitive, flexible, and quantitative methods for measuring expression levels. The first step is the isolation of RNA from a target sample. The starting material is typically total RNA isolated from human tumors or tumor cell lines. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres at al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Numerous RNA isolation kits are commercially available.

In some embodiments, the primers used for quantitative RT-PCR comprise a forward and reverse primer for each gene listed in Table 3.

In some embodiments the analytical method used for detecting at least one biomarker RNA in the methods set forth herein includes real-time quantitative RT-PCR. See Chen, C. at al. (2005) Nucl. Acids Res. 33:e179, which is incorporated herein by reference in its entirety. Although PCR can use a variety of thermostable DNA-dependent DNA polymerases, it typically employs the Taq DNA polymerase, which has a 5′-3′ nuclease activity but lacks a 3′,5′ proofreading endonuclease activity. In some embodiments, RT-PCR is done using a TaqMan® assay sold by Applied Biosystems, Inc. In a first step, total RNA is isolated from the sample. In some embodiments, the assay can be used to analyze about 10 ng of total RNA input sample, such as about 9 ng of input sample, such as about 8 ng of input sample, such as about 7 ng of input sample, such as about 6 ng of input sample, such as about 5 ng of input sample, such as about 4 ng of input sample, such as about 3 ng of input sample, such as about 2 ng of input sample, and even as little as about 1 ng of input sample containing RNA. In some embodiments, RT-PCR is done using a probe based on the Locked Nucleic Acid technology sold as Universal Probe Library (UPL) by Hoffman Laroche.

The TaqMan® assay utilizes a stern-loop primer that is specifically complementary to the 3′-end of a biomarker RNA. The step of hybridizing the stern-loop primer to the biomarker RNA is followed by reverse transcription of the biomarker RNA template, resulting in extension of the 3″ end of the primer. The result of the reverse transcription step is a chimeric (DNA) amplicon with the step-loop primer sequence at the 5′ end of the amplicon and the cDNA of the biomarker RNA at the 3′ end. Quantitation of the biomarker RNA is achieved by RT-PCR using a universal reverse primer comprising a sequence that is complementary to a sequence at the 5′ end of all stem-loop biomarker RNA primers, a biomarker RNA-specific forward primer, and a biomarker RNA sequence-specific TaqMan® probe.

The assay uses fluorescence resonance energy transfer (“FRET”) to detect and quantitate the synthesized PCR product. Typically, the TaqMan® probe comprises a fluorescent dye molecule coupled to the 5′-end and a quencher molecule coupled to the 3′-end, such that the dye and the quencher are in close proximity, allowing the quencher to suppress the fluorescence signal of the dye via FRET. When the polymerase replicates the chimeric amplicon template to which the TaqMan® probe is bound, the 5″-nuclease of the polymerase cleaves the probe, decoupling the dye and the quencher so that FRET is abolished and a fluorescence signal is generated. Fluorescence increases with each RT-PCR cycle proportionally to the amount of probe that is cleaved.

In some embodiments, quantitation of the results of RT-PCR assays is done by constructing a standard curve from a nucleic acid of known concentration and then extrapolating quantitative information for biomarker RNAs of unknown concentration. In some embodiments, the nucleic acid used for generating a standard curve is an RNA of known concentration. In some embodiments, the nucleic acid used for generating a standard curve is a purified double-stranded plasmid DNA or a single-stranded DNA generated in vitro.

In some embodiments, where the amplification efficiencies of the biomarker nucleic acids and the endogenous reference are approximately equal, quantitation is accomplished by the comparative Cₜ (cycle threshold, e.g., the number of PCR cycles required for the fluorescence signal to rise above background) method. Cₜ values are inversely proportional to the amount of nucleic acid target in a sample. In some embodiments, Cₜ values of the target RNA of interest can be compared with a control or calibrator, such as RNA from normal tissue. In some embodiments, the Cₜ values of the calibrator and the target RNA samples of interest are normalized to an appropriate endogenous housekeeping gene (see above).

In addition to the TaqMan® assays, other chemistries useful for detecting and quantitating FOR products in the methods presented herein include, but are not limited to, Nanostring technology, Molecular Beacons, Scorpion probes and SYBR Green detection.

In some embodiments, Molecular Beacons can be used to detect and quantitate FOR products. Like TaqMan® probes, Molecular Beacons use FRET to detect and quantitate a PCR product via a probe comprising a fluorescent dye and a quencher attached at the ends of the probe. Unlike TaqMan® probes, Molecular Beacons remain intact during the FOR cycles. Molecular Beacon probes form a stem-loop structure when free in solution, thereby allowing the dye and quencher to be in close enough proximity to cause fluorescence quenching. When the Molecular Beacon hybridizes to a target, the stem-loop structure is abolished so that the dye and the quencher become separated in space and the dye fluoresces. Molecular Beacons are available, e.g., from Gene Link™ (see http://www.genelink.com/newsite/products/mbintro.asp).

In some embodiments, Scorpion probes can be used as both sequence-specific primers and for FOR product detection and quantitation. Like Molecular Beacons, Scorpion probes form a stem-loop structure when not hybridized to a target nucleic acid. However, unlike Molecular Beacons, a Scorpion probe achieves both sequence-specific priming and FOR product detection. A fluorescent dye molecule is attached to the 5′-end of the Scorpion probe, and a quencher is attached to the 3′-end. The 3′ portion of the probe is complementary to the extension product of the FOR primer, and this complementary portion is linked to the 5-end of the probe by a non-amplifiable moiety. After the Scorpion primer is extended, the target-specific sequence of the probe binds to its complement within the extended amplicon, thus opening up the stem-loop structure and allowing the dye on the 5′-end to fluoresce and generate a signal. Scorpion probes are available from, e.g. Premier Biosoft International (see http://www.premierbiosoft.com/tech_notes/Scorpion.html).

In some embodiments, Nanostring technology is a system capable of highly multiplexed, direct quantification of individual mRNAs in a biological sample without the use of enzymes or amplification. It is based on color-coded “barcodes” and employs two 50-bp probes per mRNA that hybridize in solution. The Reporter Probe carries the signal; the Capture Probe allows the complex to be immobilized for data collection.

In some embodiments, RT-PCR detection is performed specifically to detect and quantify the expression of a single biomarker RNA. The biomarker RNA, in typical embodiments, is selected from a biomarker RNA capable of specifically hybridizing to a nucleic acid comprising a sequence that is identically present in one of the genes set forth in Table 3.

In various other embodiments, RT-PCR detection is utilized to detect, in a single multiplex reaction, each of 40 biomarker RNAs, or subset thereof as described herein. The biomarker RNAs, in some embodiments, are capable of specifically hybridizing to a nucleic acid comprising a sequence that is identically present in one of the forty genes listed in Table 3.

In some multiplex embodiments, a plurality of probes, such as TaqMan probes, each specific for a different RNA target, is used. In typical embodiments, each target RNA-specific probe is spectrally distinguishable from the other probes used in the same multiplex reaction.

In some embodiments, quantitation of RT-PCR products is accomplished using a dye that binds to double-stranded DNA products, such as SYBR Green. In some embodiments, the assay is the QuantiTect SYBR Green PCR assay from Qiagen. In this assay, total RNA is first isolated from a sample. Total RNA is subsequently poly-adenylated at the 3′-end and reverse transcribed using a universal primer with poly-dT at the 5-end. In some embodiments, a single reverse transcription reaction is sufficient to assay multiple biomarker RNAs. RT-PCR is then accomplished using biomarker RNA-specific primers and an miScript Universal Primer, which comprises a poly-dT sequence at the 5′-end. SYBR Green dye binds non-specifically to double-stranded DNA and upon excitation, emits light. In some embodiments, buffer conditions that promote highly-specific annealing of primers to the FOR template (e.g., available in the QuantiTect SYBR Green PCR Kit from Qiagen) can be used to avoid the formation of non-specific DNA duplexes and primer dimers that will bind SYBR Green and negatively affect quantitation. Thus, as PCR product accumulates, the signal from SYBR green increases, allowing quantitation of specific products.

RT-PCR is performed using any RT-PCR instrumentation available in the art. Typically, instrumentation used in rear-time RT-PCR data collection and analysis comprises a thermal cycler, optics for fluorescence excitation and emission collection, and optionally a computer and data acquisition and analysis software.

In some embodiments, the method of detectably quantifying one or more biomarker RNAs includes the steps of: (a) isolating total RNA; (b) reverse transcribing a biomarker RNA to produce a cDNA that is complementary to the biomarker RNA; (c) amplifying the cDNA from step (b); and (d) detecting the amount of a biomarker RNA with RT-PCR.

As described above, in some embodiments, the RT-PCR detection is performed using a FRET probe, which includes, but is not limited to, a TaqMan® probe, a Nanostring probe set, a Molecular beacon probe and a Scorpion probe. In some embodiments, the RT-PCR detection and quantification is performed with a TaqMan® probe, i.e., a linear probe that typically has a fluorescent dye covalently bound at one end of the DNA and a quencher molecule covalently bound at the other end of the DNA. The FRET probe comprises a base sequence that is complementary to a region of the cDNA such that, when the FRET probe is hybridized to the cDNA, the dye fluorescence is quenched, and when the probe is digested during amplification of the cDNA, the dye is released from the probe and produces a fluorescence signal. In such embodiments, the amount of biomarker RNA in the sample is proportional to the amount of fluorescence measured during cDNA amplification.

The TaqMan® probe typically comprises a region of contiguous nucleotides comprising a base sequence that is complementary to a region of a biomarker RNA or its complementary cDNA that is reverse transcribed from the biomarker RNA template (i.e., the sequence of the probe region is complementary to or identically present in the biomarker RNA to be detected) such that the probe is specifically hybridizable to the resulting PCR amplicon. In some embodiments, the probe comprises a region of at least 6 contiguous nucleotides having a base sequence that is fully complementary to or identically present in a region of a cDNA that has been reverse transcribed from a biomarker RNA template, such as comprising a region of at least 8 contiguous nucleotides, or comprising a region of at least 10 contiguous nucleotides, or comprising a region of at least 12 contiguous nucleotides, or comprising a region of at least 14 contiguous nucleotides, or even comprising a region of at least 16 contiguous nucleotides having a base sequence that is complementary to or identically present in a region of a cDNA reverse transcribed from a biomarker RNA to be detected.

Preferably, the region of the cDNA that has a sequence that is complementary to the TaqMan® probe sequence is at or near the center of the cDNA molecule. In some embodiments, there are independently at least 2 nucleotides, such as at least 3 nucleotides, such as at least 4 nucleotides, such as at least 5 nucleotides of the cDNA at the 5-end and at the 3′-end of the region of complementarity.

In typical embodiments, all biomarker RNAs are detected in a single multiplex reaction. In these embodiments, each TaqMan® probe that is targeted to a unique cDNA is spectrally distinguishable when released from the probe. Thus, each biomarker RNA is detected by a unique fluorescence signal.

In some embodiments, expression levels may be represented by gene transcript numbers per nanogram of cDNA. To control for variability in cDNA quantity, integrity and the overall transcriptional efficiency of individual primers, RT-PCR data can be subjected to standardization and normalization against one or more housekeeping genes as has been previously described. See e.g., Rubie at al., Mol. Cell. Probes 19(2):101-9 (2005).

Appropriate genes for normalization in the methods described herein include those as to which the quantity of the product does not vary between different cell types, cell lines or under different growth and sample preparation conditions. In some embodiments, endogenous housekeeping genes useful as normalization controls in the methods described herein include, but are not limited to, ACTB, BAT1, EDS, B2M, TBP, U6 snRNA, RNU44, RNU 48, and U47. In typical embodiments, the at least one endogenous housekeeping gene for use in normalizing the measured quantity of RNA is selected from ACTB, BAT1, EDS, B2M, TBP, U6 snRNA, U6 snRNA, RNU44, RNU 48, and U47. In some embodiments, normalization to the geometric mean of two, three, four or more housekeeping genes is performed. In some embodiments, one housekeeping gene is used for normalization. In some embodiments, two, three, four or more housekeeping genes are used for normalization.

In some embodiments, labels that can be used on the FRET probes include calorimetric and fluorescent labels such as Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610. Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5°,7′-Tetrabromosulfonefluorescein, and TET.

Specific examples of fluorescently labeled ribonucleotides useful in the preparation of RT-PCR probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences (GE Healthcare), such as Cy3-UTP and Cy5-UTP.

Examples of fluorescently labeled deoxyribonucleotides useful in the preparation of RT-PCR probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 6501665-14-dUTP; Alexa Fluor 488-7-OBEA-dCTP, Alexa Fluor 546-16-OBEA-dCTP, Alexa fluor 594-7-OBEA-dCTP, Alexa Fluor 647-12-OBEA-dCTP. Fluorescently labeled nucleotides are commercially available and can be purchased from, e.g., Invitrogen.

In some embodiments, dyes and other moieties, such as quenchers, are introduced into nucleic acids used in the methods described herein, such as FRET probes, via modified nucleotides. A “modified nucleotide” refers to a nucleotide that has been chemically modified, but still functions as a nucleotide. In some embodiments, the modified nucleotide has a chemical moiety, such as a dye or quencher, covalently attached, and can be introduced into an oligonucleotide, for example, by way of solid phase synthesis of the oligonucleotide. In other embodiments, the modified nucleotide includes one or more reactive groups that can react with a dye or quencher before, during, or after incorporation of the modified nucleotide into the nucleic acid. In specific embodiments, the modified nucleotide is an amine-modified nucleotide, i.e., a nucleotide that has been modified to have a reactive amine group. In some embodiments, the modified nucleotide comprises a modified base moiety, such as uridine, adenosine, guanosine, and/or cytosine. In specific embodiments, the amine-modified nucleotide is selected from 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP. In some embodiments, nucleotides with different nucleobase moieties are similarly modified, for example, 5-(3-aminoallyl)-GTP instead of 5-(3-aminoallyl)-UTP. Many amine modified nucleotides are commercially available from, e.g., Applied Biosystems, Sigma, Jena Bioscience and TriLink.

In some embodiments, the methods of detecting at least one biomarker RNA described herein employ one or more modified oligonucleotides, such as oligonucleotides comprising one or more affinity-enhancing nucleotides. Modified oligonucleotides useful in the methods described herein include primers for reverse transcription, PCR amplification primers, and probes. In some embodiments, the incorporation of affinity-enhancing nucleotides increases the binding affinity and specificity of an oligonucleotide for its target nucleic acid as compared to oligonucleotides that contain only deoxyribonucleotides, and allows for the use of shorter oligonucleotides or for shorter regions of complementarity between the oligonucleotide and the target nucleic acid.

In some embodiments, affinity-enhancing nucleotides include nucleotides comprising one or more base modifications, sugar modifications and/or backbone modifications.

In some embodiments, modified bases for use in affinity-enhancing nucleotides include 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine.

In some embodiments, affinity-enhancing modifications include nucleotides having modified sugars such as 2′-substituted sugars, such as 2-O-alkyl-ribose sugars, 2-amino-deoxyribose sugars, 2-fluoro-deoxyribose sugars, 2′-fluoro-arabinose sugars, and 2′-O-methoxyethyl-ribose (2′MOE) sugars. In some embodiments, modified sugars are arabinose sugars, or d-arabino-hexitol sugars.

In some embodiments, affinity-enhancing modifications include backbone modifications such as the use of peptide nucleic acids (e.g., an oligomer including nucleobases linked together by an amino acid backbone). Other backbone modifications include phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates; carboxymethyl esters, methylphosphorothioate, phosphorodithioate, p-ethoxy, and combinations thereof.

In some embodiments, the oligomer includes at least one affinity-enhancing nucleotide that has a modified base, at least nucleotide (which may be the same nucleotide) that has a modified sugar, and at least one internucleotide linkage that is non-naturally occurring.

In some embodiments, the affinity-enhancing nucleotide contains a locked nucleic acid (“LNA”) sugar, which is a bicyclic sugar. In some embodiments, an oligonucleotide for use in the methods described herein comprises one or more nucleotides having an LNA sugar. In some embodiments, the oligonucleotide contains one or more regions consisting of nucleotides with LNA sugars. In other embodiments, the oligonucleotide contains nucleotides with LNA sugars interspersed with deoxyribonucleotides. See, e.g., Frieden, M. et al., (2008) Curr. Pharm. Des. 14(11): 1138-1142.

The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art.

In addition, a person skilled in the art will appreciate that a number of methods can be used to determine the amount of a protein product of the biomarker of the invention, including immunoassays such as Western blots, ELISA, and immunoprecipitation followed by SDS-PAGE and immunocytochemistry.

Accordingly, in another embodiment, an antibody is used to detect the polypeptide products of the forty biomarkers listed in Table 3. In another embodiment, the sample comprises a tissue sample. In a further embodiment, the tissue sample is suitable for immunohistochemistry.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Conventional techniques of molecular biology, microbiology and recombinant DNA techniques are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Harnes & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995).

For example, antibodies having specificity fore specific protein, such as the protein product of a biomarker, may be prepared by conventional methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse at al., Science 246:1275 (1989)). Hybridoma cells can be screened immunochemically for production of antibodies specifically reactive with the peptide and the monoclonal antibodies can be isolated.

In some embodiments, recombinant antibodies are provided that specifically bind protein products of the forty genes listed in Table 3. Recombinant antibodies include, but are not limited to, chimeric and humanized monoclonal antibodies, comprising both human and non-human portions, single-chain antibodies and multi-specific antibodies. A chimeric antibody is a molecule in which different portions are derived from different animal species, such as those having a variable region derived from a murine monoclonal antibody (mAb) and a human immunoglobulin constant region. (See, e.g., Cabilly at al., U.S. Pat. No. 4,816,567; and Boss et al., U.S. Pat. No. 4,816,397, which are incorporated herein by reference in their entirety.) Single-chain antibodies have an antigen binding site and consist of single polypeptides. They can be produced by techniques known in the art, for example using methods described in Ladner et. al U.S. Pat. No. 4,946,778 (which is incorporated herein by reference in its entirety); Bird et al., (1988) Science 242:423-426; Whitlow et al., (1991) Methods in Enzymology 2:1-9; Whitlow et al., (1991) Methods in Enzymology 2:97-105; and Huston et al., (1991) Methods in Enzymology Molecular Design and Modeling: Concepts and Applications 203:46-88. Multi-specific antibodies are antibody molecules having at least two antigen-binding sites that specifically bind different antigens. Such molecules can be produced by techniques known in the art, for example using methods described in Segal, U.S. Pat. No. 4,676,980 (the disclosure of which is incorporated herein by reference in its entirety); Holliger et al., (1993) Proc. Natl. Acad. Sci. USA 90:6444-6448; Whitlow et al., (1994) Protein Eng 7:1017-1026 and U.S. Pat. No. 6,121,424.

Monoclonal antibodies directed against any of the expression products of the genes listed in Table 3 can be identified and isolated by screening a recombinant combinatorial immunoglobulin library (e.g., an antibody phage display library) with the polypeptide(s) of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; POT Publication No. WO 92/18619; PCT Publication No. WO 91/17271; PCT Publication No. WO 92/20791; POT Publication No. WO 92/15679; POT Publication No. WO 93/01288; POT Publication No. WO 92/01047; POT Publication No. WO 92/09690; POT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734.

Humanized antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.) Humanized monoclonal antibodies can be produced by recombinant DNA techniques known in the art, for example using methods described in POT Publication No. WO 87/02671; European Patent Application 184,187; European Patent Application 171,496; European Patent Application 173,494; POT Publication No. WO 86/01533; U.S. Pat. No. 4,816,567; European Patent Application 125,023; Better at al. (1988) Science 240:1041-1043; Liu at al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw at al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones at al. (1986) Nature 321:552-525; Verhoeyan et al. (1988) Science 239:1534; and Beidler et al. (1988) J. Immunol. 141:4053-4060.

In some embodiments, humanized antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a protein product. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) hit. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc, (Fremont, Calif.), can be engaged to provide human antibodies directed against a selected antigen using technology similar to that described above.

Antibodies may be isolated after production (e.g., from the blood or serum of the subject) or synthesis and further purified by well-known techniques. For example, IgG antibodies can be purified using protein A chromatography. Antibodies specific for a protein can be selected or (e.g., partially purified) or purified by, e.g., affinity chromatography. For example, a recombinantly expressed and purified (or partially purified) expression product may be produced, and covalently or non-covalently coupled to a solid support such as, for example, a chromatography column. The column can then be used to affinity purify antibodies specific for the protein products of the genes listed in Tables 3 and 4 from a sample containing antibodies directed against a large number of different epitopes, thereby generating a substantially purified antibody composition, i.e., one that is substantially free of contaminating antibodies. By a substantially purified antibody composition it is meant, in this context, that the antibody sample contains at most only 30% (by dry weight) of contaminating antibodies directed against epitopes other than those of the protein products of the genes listed in Tables 3 and 4, and preferably at most 20%, yet more preferably at most 10%, and most preferably at most 5% (by dry weight) of the sample is contaminating antibodies. A purified antibody composition means that at least 99% of the antibodies in the composition are directed against the desired protein.

In some embodiments, substantially purified antibodies may specifically bind to a signal peptide, a secreted sequence, an extracellular domain, a transmembrane or a cytoplasmic domain or cytoplasmic membrane of a protein product of one of the genes listed in Table 3. In an embodiment, substantially purified antibodies specifically bind to a secreted sequence or an extracellular domain of the amino acid sequences of a protein product of one of the genes listed in Table 3, or subset thereof.

In some embodiments, antibodies directed against a protein product of one of the genes listed in Table 3 can be used to detect the protein products or fragment thereof (e.g., in a cellular lysate or cell supernatant) in order to evaluate the level and pattern of expression of the protein. Detection can be facilitated by the use of an antibody derivative, which comprises an antibody coupled to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

A variety of techniques can be employed to measure expression levels of each of the forty, and optional additional, genes given a sample that contains protein products that bind to a given antibody. Examples of such formats include, but are not limited to, enzyme immunoassay (EIA), radioimmunoassay (RIA), Western blot analysis and enzyme linked immunoabsorbent assay (ELISA). A skilled artisan can readily adapt known protein/antibody detection methods for use in determining protein expression levels of the forty, and optional additional products of the genes listed in Table 3.

In one embodiment, antibodies, or antibody fragments or derivatives, can be used in methods such as Western blots or immunofluorescence techniques to detect the expressed proteins. In some embodiments, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of the prognostic markers. In some embodiments, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art (Ausubel, supra, unit 10.1-10.6). A two-site, monoclonal-based immunoassay utilizing antibodies reactive to two non-interfering epitopes is preferred, but a competitive binding assay may be employed (Pound (1998) Immunochemical Protocols, Humana Press, Totowa N.J.).

Numerous labels are available which can be generally grouped into the following categories:
(a) Radioisotopes, such as ³⁶S, ¹⁴O, ¹²⁵I, ³H, and ¹³¹I. The antibody variant can be labeled with the radioisotope using the techniques described in Current Protocols in Immunology, vol 1-2, Coligen et al., Ed., Wiley-Interscience, New York, Pubs. (1991) for example and radioactivity can be measured using scintillation counting.
(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques disclosed in Current Protocols in Immunology, supra, for example. Fluorescence can be quantified using a fluorimeter.
(c) Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzyme. (Ed. J. Langone & H. Van Vunakis), Academic press, New York, 73: 147-166 (1981).

In some embodiments, a detection label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g. digoxin) and one of the different types of labels mentioned above is conjugated with an anti-hapten antibody (e.g. anti-digoxin antibody). In some embodiments, the antibody need not be labeled, and the presence thereof can be detected using a labeled antibody, which binds to the antibody.

The present application provides compositions useful in detecting changes in the expression levels of the 40 genes listed in Table 3. Accordingly in one embodiment, the application provides a composition comprising a plurality of isolated nucleic acid sequences wherein each isolated nucleic acid sequence hybridizes to:
(a) an RNA product of one of the 40 genes listed in Table 3; and/or
(b) a nucleic acid complementary to a),
   wherein the composition is used to measure the level of expression of the 40 genes.

In some embodiments, the application provides compositions comprising 40 forward and 40 reverse primers for amplifying a region of each gene listed in Table 3.

In a further aspect, the application also provides an array that is useful in detecting the expression levels of the 40 genes listed in Table 3, or subset thereof. Accordingly, in one embodiment, the application provides an array comprising for each gene shown in Table 3 one or more nucleic acid probes complementary and hybridizable to an expression product of the gene.

In yet another aspect, the application also provides for kits used to prognose or classify a subject with NSCLC into a good survival group or a poor survival group that includes detection agents that can detect the expression products of the biomarkers. Accordingly, in one embodiment, the application provides a kit to prognose or classify a subject with early stage NSCLC comprising detection agents that can detect the expression products of 40 biomarkers, wherein the 40 biomarkers comprise 40 genes in Table 3. In another embodiment, kits for classifying a subject comprise detection agents that can detect the expression of 41, 42, or 43 biomarkers, wherein 40 biomarkers comprise the 40 genes in Table 3.

The materials and methods of the present disclosure are ideally suited for preparation of kits produced in accordance with well known procedures. Kits, may comprise containers, each with one or more of the various reagents (sometimes in concentrated form), for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more primer complexes (e.g., appropriate length poly(T) or random primers linked to a promoter reactive with the RNA polymerase). A set of instructions will also typically be included.

In some embodiments, a kit may comprise a plurality of reagents, each of which is capable of binding specifically with a target nucleic acid or protein. Suitable reagents for binding with a target protein include antibodies, antibody derivatives, antibody fragments, and the like. Suitable reagents for binding with a target nucleic acid (e.g. a genomic DNA, an mRNA, a spliced mRNA, a cDNA, or the like) include complementary nucleic acids. For example, nucleic acid reagents may include oligonucleotides (labeled or non-labeled) fixed to a substrate, labeled oligonucleotides not bound with a substrate, pairs of FOR primers, molecular beacon probes, and the like.

In some embodiments, kits may comprise additional components useful for detecting gene expression levels. By way of example, kits may comprise fluids (e.g. SSC buffer) suitable for annealing complementary nucleic acids or for binding an antibody with a protein with which it specifically binds, one or more sample compartments, a material which provides instruction for detecting expression levels, and the like.

In some embodiments, kits for use in the RT-PCR methods described herein comprise one or more target RNA-specific FRET probes and one or more primers for reverse transcription of target RNAs or amplification of cDNA reverse transcribed therefrom.

In some embodiments, one or more of the primers is “linear”, A “linear” primer refers to an oligonucleotide that is a single stranded molecule, and typically does not comprise a short region of, for example, at least 3, 4 or 5 contiguous nucleotides, which are complementary to another region within the same oligonucleotide such that the primer forms an internal duplex. In some embodiments, the primers for use in reverse transcription comprise a region of at least 4, such as at least 5, such as at least 6, such as at least 7 or more contiguous nucleotides at the 3′-end that has a base sequence that is complementary to region of at least 4, such as at least 5, such as at least 6, such as at least 7 or more contiguous nucleotides at the 5′-end of a target RNA.

In some embodiments, the kit further comprises one or more pairs of linear primers (a “forward primer” and a “reverse primer”) for amplification of a cDNA reverse transcribed from a target RNA. Accordingly, in some embodiments, the forward primer comprises a region of at least 4, such as at least 5, such as at least 6, such as at least 7, such as at least 8, such as at least 9, such as at least 10 contiguous nucleotides having a base sequence that is complementary to the base sequence of a region of at least 4, such as at least 5, such as at least 6, such as at least 7, such as at least 8, such as at least 9, such as at least 10 contiguous nucleotides at the 5-end of a target RNA. Furthermore, in some embodiments, the reverse primer comprises a region of at least 4, such as at least 5, such as at least 6, such as at least 7, such as at least 8, such as at least 9, such as at least 10 contiguous nucleotides having a base sequence that is complementary to the base sequence of a region of at least 4, such as at least 5, such as at least 6, such as at least 7, such as at least 8, such as at least 9, such as at least 10 contiguous nucleotides at the 3′-end of a target RNA.

In some embodiments, the kit comprises at least a first set of primers for amplification of a cDNA that is reverse transcribed from a target RNA capable of specifically hybridizing to a nucleic acid comprising a sequence identically present in one of the genes listed in Table 3. In some embodiments, the kit comprises at least forty sets of primers, each of which is for amplification of a different target RNA capable of specifically hybridizing to a nucleic acid comprising a sequence identically present in a different gene listed in Table 3. In some embodiments, the kit comprises at least one set of primers that is capable of amplifying more than one cDNA reverse transcribed from a target RNA in a sample.

In some embodiments, probes and/or primers for use in the compositions described herein comprise deoxyribonucleotides. In some embodiments, probes and/or primers for use in the compositions described herein comprise deoxyribonucleotides and one or more nucleotide analogs, such as LNA analogs or other duplex-stabilizing nucleotide analogs described above. In some embodiments, probes and/or primers for use in the compositions described herein comprise all nucleotide analogs. In some embodiments, the probes and/or primers comprise one or more duplex-stabilizing nucleotide analogs, such as LNA analogs, in the region of complementarity.

In some embodiments, the compositions described herein also comprise probes, and in the case of RT-PCR, primers, that are specific to one or more housekeeping genes for use in normalizing the quantities of target RNAs. Such probes (and primers) include those that are specific for one or more products of housekeeping genes selected from ACTB, BAT1, EDS, B2M, TBP, U6 snRNA, RNU44, RNU 48, and U47.

In some embodiments, the kits for use in real time RT-PCR methods described herein further comprise reagents for use in the reverse transcription and amplification reactions. In some embodiments, the kits comprise enzymes such as reverse transcriptase, and a heat stable DNA polymerase, such as Taq polymerase. In some embodiments, the kits further comprise deoxyribonucleotide triphosphates (dNTP) for use in reverse transcription and amplification. In further embodiments, the kits comprise buffers optimized for specific hybridization of the probes and primers.

In some embodiments, kits are provided containing antibodies to each of the protein products of the genes listed in Table 3, conjugated to a detectable substance, and instructions for use. Kits may comprise an antibody, an antibody derivative, or an antibody fragment, which binds specifically with a marker protein, or a fragment of the protein. Such kits may also comprise a plurality of antibodies, antibody derivatives, or antibody fragments wherein the plurality of such antibody agents binds specifically with a marker protein, or a fragment of the protein.

In some embodiments, kits may comprise antibodies such as a labeled or labelable antibody and a compound or agent for detecting protein in a biological sample; means for determining the amount of protein in the sample; means for comparing the amount of protein in the sample with a standard; and instructions for use. Such kits can be supplied to detect a single protein or epitope or can be configured to detect one of a multitude of epitopes, such as in an antibody detection array. Arrays are described in detail herein for nucleic acid arrays and similar methods have been developed for antibody arrays.

A person skilled in the art will appreciate that a number of detection agents can be used to determine the expression of the biomarkers. For example, to detect RNA products of the biomarkers, probes, primers, complementary nucleotide sequences or nucleotide sequences that hybridize to the RNA products can be used. To detect protein products of the biomarkers, ligands or antibodies that specifically bind to the protein products can be used.

Accordingly, in one embodiment, the detection agents are probes that hybridize to the 40 biomarkers. In another embodiment, the detection agents are forward and reverse primers that amplify a region of each of the 40 genes listed in Table 3.

A person skilled in the art will appreciate that the detection agents can be labeled.

The label is preferably capable of producing, either directly or indirectly, a detectable signal. For example, the label may be radio-opaque or a radioisotope, such as ³H, ¹⁴O, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The kit can also include a control or reference standard and/or instructions for use thereof. In addition, the kit can include ancillary agents such as vessels for storing or transporting the detection agents and/or buffers or stabilizers.

In a further aspect, the application provides computer programs and computer implemented products for carrying out the methods described herein. Accordingly, in one embodiment, the application provides a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the methods described herein.

In another embodiment, the application provides a computer implemented product for predicting a prognosis or classifying a subject with NSCLC comprising:

(a) a means for receiving values corresponding to a subject expression profile in a subject sample; and

(b) a database comprising a reference expression profile associated with a prognosis, wherein the subject biomarker expression profile and the biomarker reference profile each has forty values, each value representing the expression level of a biomarker, wherein each biomarker corresponds to one gene in Table 3;

wherein the computer implemented product uses the biomarker reference expression profile to evaluate the subject biomarker expression profile, to thereby predict a prognosis or classify the subject.

Another aspect relates to computer readable mediums such as CD-ROMs. In one embodiment, the application provides computer readable medium having stored thereon a data structure for storing a computer implemented product described herein.

In one embodiment, the data structure is capable of configuring a computer to respond to queries based on records belonging to the data structure, each of the records comprising:

(a) a value that identifies a biomarker reference expression profile of the 40 genes in Table 3 or subset thereof;

(b) a value that identifies the probability of a prognosis associated with the biomarker reference expression profile.

In some embodiments, the application provides a computer implemented product comprising

(a) a means for receiving values corresponding to relative expression levels in a subject, of at least 40 biomarkers comprising the forty genes in Table 3;

(b) an algorithm for calculating a risk score based on the relative expression levels of the at least 40 biomarkers;

(c) an output that displays the risk score; and, optionally,

(d) an output that displays a prognosis based on the risk score.

The above disclosure generally describes the present invention. A more complete understanding can be obtained by reference to the following specific example. This example is described solely for the purpose of illustration and is not intended to limit the scope of the invention. Changes in form and substitution of equivalents are contemplated as circumstances might suggest or render expedient. Although specific terms have been employed herein, such terms are intended in a descriptive sense and not for purposes of limitation.

The following non-limiting example is illustrative of the present invention.

### Example 1

### Gene Signature Model Development

To generate the gene expression signature, normalized whole genome gene expression data based on microarray analysis was downloaded from publicly available databases: Directors Challenge (Affymetrix, Shedden 2008), Duke (Affymetrix, Potti 2006), University of Michigan (Affymetrix, Raponi 2006) and NLCI (Agilent, Roepman et al., CCR 2009). After applying rigorous inclusion and exclusion criteria, the gene expression data of 579 NSCLC patient samples were pre-processed by log transformation and sample scaling to a zero median and unit variance.

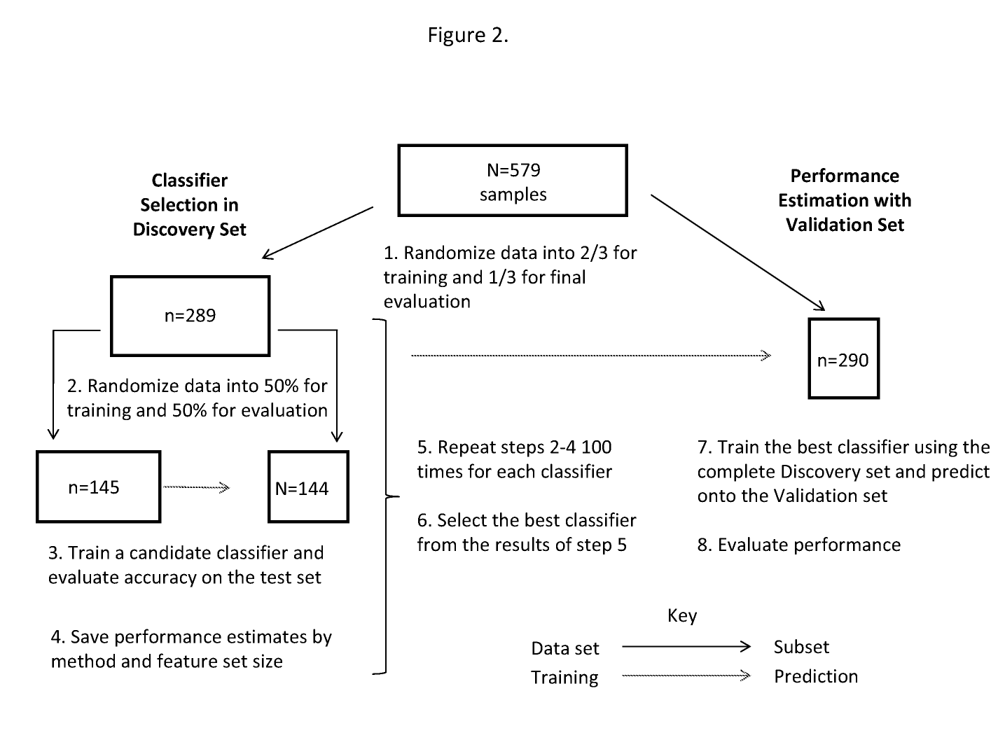

The 579 samples were randomized into 2 groups: One group, composed of 289 samples, was used as a Discovery set and helped select the classifier, the other, composed of 290 samples served as a Validation set, helping in evaluating the performance of the classifier. In the Discovery set, data was further randomized 1:1 into a training set (145 samples) and a validation set (144 samples). A candidate classifier was then trained and evaluated for accuracy on the test set. Performance estimates were then saved by method and feature set size. Steps involving 1:1 randomization, training, and evaluation were repeated 100 times for each classifier and the best classifier was selected as a result. Following that, the best classifier was trained using the complete Discovery set and then tested for prognostic power in the Validation set. See FIG. 2.

Prediction analysis was performed by evaluating the expression status of the each of the genes in the signature identified using the nearest template prediction (NTP) method as implemented in the NearestTemplatePrediction module of the GenePattern analysis toolkit. A hypothetical training sample serving as the template of outcome was defined as a vector having the same length as the predictive signature. In this template, a value of 1 was assigned to “poor” outcome-correlated genes and a value of −1 was assigned to “good” outcome-correlated genes, and then each gene was weighted by the absolute value of the corresponding Cox statistic.

To identify technical variability among the different sites, platforms and protocols, Principal Component Analysis (PCA) was performed. First, the three Affymetrix data sets were combined by probeset identifiers. PCA revealed a site bias, which was confounded with the histology of the samples. Probeset specific adjustment factors were estimated after stratification by site and histology, and applied. This accounted for the technical bias but did not greatly reduce the biological variance. The Agilent-based dataset was then added by mean collapsing the Affymetrix and Agilent data by manufacturer supplied Entrez Gene identifiers. Again, gene specific adjustment factors were estimated after stratification by Affymetrix, Agilent, and histology, to account for the technical variation between the two platforms. The final data set did not reveal any technical bias in PCA and maintained the primary biological variance associated with histology. A total of 7515 individual genes were represented in the final combined data matrix.

Two different approaches were used to attempt to develop a signature: Nearest Template Prediction, or NTP, and Lasso regression. See Hoshida Y, NEJM, 2008, 359:1995-2004 and Tibshirani, R, Statistics in Med., 1997, 16:385-395. Using NTP, the CoxPH statistic for all genes in a possible signature was calculated. Genes were then ranked by the absolute value of the CoxPH statistic and the top N genes were selected. Test cases were scored with the inner product of the vector of CoxPH statistics and relative expression values of the sample. In a second experiment, Lasso regression was used to model gene expression modules.²¹ Test samples were then scored using the inner product of coefficients from the model and module.

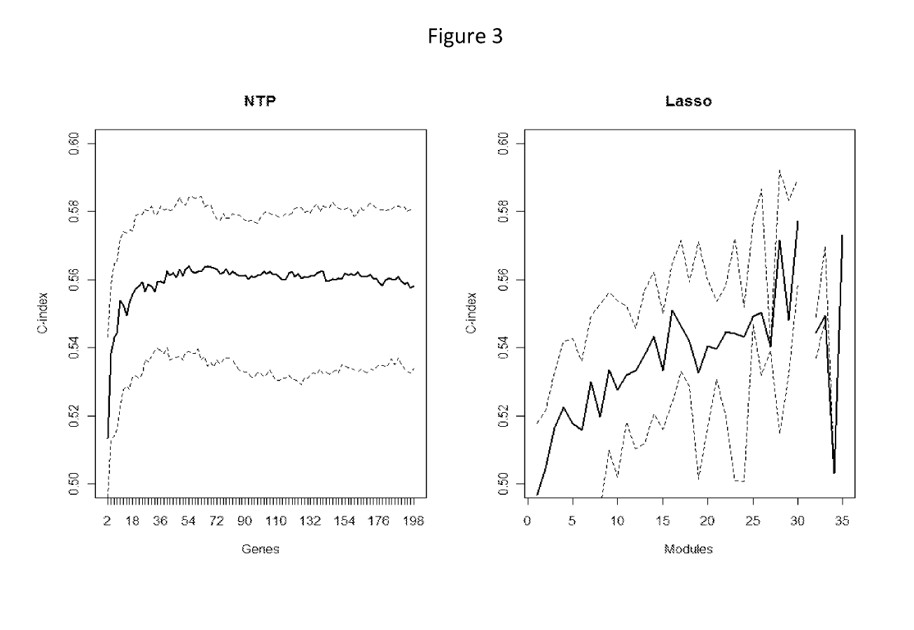

The Concordance Index was used to estimate classification performance (Harrell F E, Jr. et al. JAMA 1982; 247(18):2543-2546) and served as a basis for choosing a 40 gene-signature identified in the NTP model for final evaluation (FIG. 3), Lasso regression modelling was inconclusive and did not lead to the identification of an optimal module.

All statistical analyses were performed using the R statistical package and the ‘penalized’ library²².

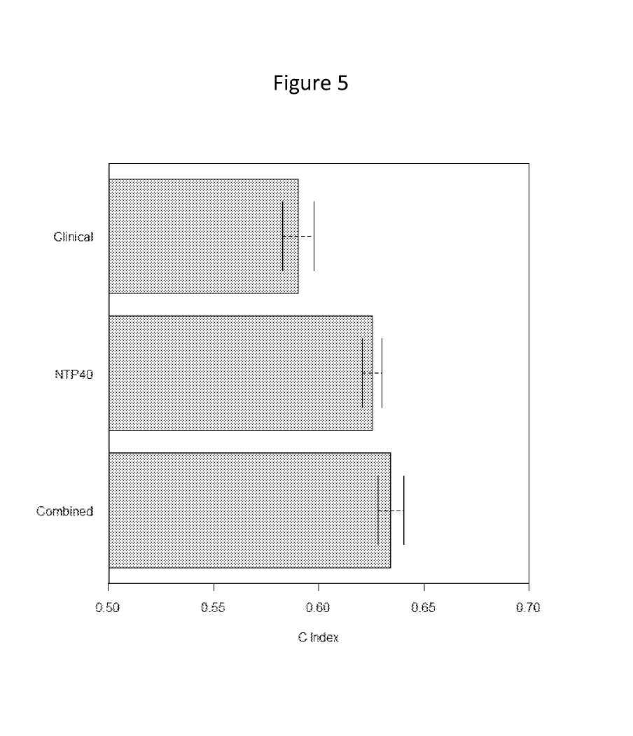

Performance of a 40-gene signature was evaluated on the Validation set by first calculating risk scores for the samples in the Validation set and then using the Concordance index to assess the prognostic power of the proposed signature relative to clinical assessment alone. The gene signature provides a significant increase in prognostic power relative to prognosis based on clinical data alone. See FIG. 5, C-index (clinical data)=0.59+/−0.04 versus C-index (NTP40 signature)=0.63+/−0.04. The risk scores were calculated by taking the sum of the inner product of the reference values of the 40 genes (see Table 3) and the relative expression levels for each of the 40 genes.

### Prognostic Modeling of Signature Genes

A gene expression signature is thought to represent the altered key pathways in carcinogenesis and thus is able to predict patients outcome. However, being able to faithfully represent the altered key pathways, the signature must be generated from genome-wide gene expression data. The present study used all information generated by Affymetrix U133A, Affymetrix U133 Plus2, or Agilent chips, on NSCLC samples from 4 patient cohorts to derive a 40-gene signature. The 40-gene signature was able to identify 41% ( 83/202) stage IB-II NSCLC patients that had a relative good outcome. Multivariate analysis indicated that the 40-gene signature was an independent prognostic factor. Moreover, its independent prognostic effect has been validated in silico on 290 NSCLC samples without adjuvant chemo- or radiotherapy from DC, NLCI, Duke, and the University of Michigan.

Adjuvant chemotherapy for completely resected early stage NSCLC was a research question until the results of a series of positive trials^{2,4}, including BR.10³, were published. However, whether chemotherapy played a beneficial role in stage IB remained to be clarified²⁻⁶. The present study showed that the stage IB patients were potentially able to be separated into groups with significantly different mortality rates, or hazard rate.

Another significance of the present study was that the signature was able to identify a subgroup of patients from stage II (23%, 23/81), with a very low risk of mortality and therefore might not benefit from treatment or intervention beyond surgery.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Sample Characteristics

| | | | | | | Vali- | Com- |
|---|---|---|---|---|---|---|---|
| | DC | NLCI | Duke | Michigan | Discovery | dation | bined |
| N | 249 | 148 | 76 | 106 | 290 | 289 | 579 |
| Stage | | | | | | | |
| IA | 90 | 34 | 33 | 27 | 88 | 96 | 184 |
| IB | 107 | 66 | 27 | 46 | 121 | 125 | 246 |
| IIA | 16 | 6 | 4 | 6 | 18 | 14 | 32 |
| IIB | 36 | 42 | 12 | 27 | 54 | 63 | 117 |
| Histology | | | | | | | |
| Adeno | 249 | 56 | 36 | 0 | 171 | 170 | 341 |
| SQC | 0 | 92 | 40 | 106 | 119 | 119 | 238 |
| Age | | | | | | | |
| 25% | 59 | 56 | 57 | 62 | 59 | 58 | 234 |
| Median | 65 | 64 | 66 | 70 | 66 | 65 | 265 |
| 75% | 73 | 70 | 70 | 75 | 72 | 73 | 288 |
| Gender | | | | | | | |
| Female | 109 | NA | 31 | 37 | 92 | 85 | 177 |
| Male | 140 | NA | 45 | 69 | 124 | 130 | 254 |

TABLE 2
Multivariable analysis of validation set.

| | HR | CI | p-value |
|---|---|---|---|
| NTP40 | 1.45 | 1.2-1.8 | 0.0006 |
| Age | 1.02 | 1.0-1.0 | 0.0292 |
| Histology | 1.09 | 0.5-2.3 | 0.8160 |
| Stage | | | |
| IB | 1.56 | 0.9-2.6 | 0.0803 |
| IIA | 3.00 | 1.5-6.2 | 0.0029 |
| IIB | 2.57 | 1.5-4.4 | 0.0008 |

N = 290
Events = 123

| | | | |
|---|---|---|---|
| NTP40 | 1.42 | 1.1-1.8 | 0.0044 |
| Age | 1.02 | 1.0-1.0 | 0.0282 |
| Histology | 0.67 | 0.2-1.8 | 0.4342 |
| Stage | | | |
| IB | 1.48 | 0.9-2.5 | 0.1483 |
| IIA | 3.40 | 1.6-7.2 | 0.0013 |
| IIB | 2.39 | 1.3-4.3 | 0.0036 |
| Gender | 0.91 | 0.6-1.4 | 0.6385 |

N = 216
Events = 105
Multivariable analysis of the validation set demonstrates that the NTP40
predictor carries independent prognostic information with respect to
standard clinical variables.
Both models were stratified by site.

TABLE 3
Genes included in the NTP40 classifier.

| Entrez | Name | Description | weight |
|---|---|---|---|
| 205 | AK3L1 | denylate kinase 3-like 1 | 4.353761 |
| 4150 | MAZ | MYC-associated zinc finger protein | 4.123683 |
| 57644 | MYH7B | myosin, heavy polypeptide 7B, cardiac muscle | 3.874657 |
| 4613 | MYCN | v-myc myelocytomatosis viral related oncogene | 3.767398 |
| 10785 | WDR4 | WD repeat domain 4 | 3.681693 |
| 55777 | MBD5 | methyl-CpG binding domain protein 5 | 3.646531 |
| 8260 | ARD1A | ARD1 homolog A, N-acetyltransferase | 3.579068 |
| 79175 | ZNF343 | zinc finger protein 343 | 3.394088 |
| 83729 | INHBE | inhibin, beta E | 3.388262 |
| 9391 | WDR39 | WD repeat domain 39 | 3.325111 |
| 6865 | TACR2 | tachykinin receptor 2 | 3.310073 |
| 441601 | LOC441601 | septin 7 pseudogene | 3.304427 |
| 6566 | SLC16A1 | solute carrier family 16, member 1 | 3.299845 |
| 10998 | SLC27A5 | solute carrier family 27 (fatty acid transporter) | 3.282334 |
| 3768 | KCNJ12 | potassium inwardly-rectifying channel | 3.26168 |
| 10714 | POLD3 | polymerase (DNA-directed), delta 3 | 3.251923 |
| 5119 | RPA3 | replication protein A3, 14 kDa | 3.247453 |
| 4762 | NEUROG1 | neurogenin 1 | 3.226022 |
| 23658 | LSM5 | LSM5 homolog, U6 small nuclear RNA associated | 3.210248 |
| 10261 | IGSF6 | immunoglobulin superfamily, member 6 | −3.19966 |
| 66008 | TRAK2 | trafficking protein, kinesin binding 2 | −3.21156 |
| 23180 | RAFTLIN | raft-linking protein | −3.21268 |
| 80342 | TRAF3IP3 | TRAF3 interacting protein 3 | −3.24484 |
| 91353 | CTA-246H3.1 | similar to omega protein | −3.26956 |
| 608 | TNFRSF17 | tumor necrosis factor receptor superfamily, member 17 | −3.2696 |
| 3500 | IGHG1 | anti-rabies SO57 immunoglobulin heavy chain | −3.28005 |
| 9693 | RAPGEF2 | Rap guanine nucleotide exchange factor (GEF) 2 | −3.28713 |
| 27334 | P2RY10 | purinergic receptor P2Y, G-protein coupled, 10 | −3.30631 |
| 8837 | CFLAR | CASP8 and FADD-like apoptosis regulator | −3.33254 |
| 57178 | RAI17 | retinoic acid induced 17 | −3.34212 |
| 57509 | MTUS1 | mitochondrial tumor suppressor 1 | −3.34328 |
| 1258 | CNGB1 | cyclic nucleotide gated channel beta 1 | −3.35761 |
| 54704 | PPM2C | protein phosphatase 2C, magnesium-dependent | −3.3635 |
| 9166 | EBAG9 | estrogen receptor binding site associated, antigen, 9 | −3.36452 |
| 695 | BTK | Bruton agammaglobulinemia tyrosine kinase | −3.3774 |
| 6726 | SRP9 | signal recognition particle 9 kDa | −3.48223 |
| 2034 | EPAS1 | endothelial PAS domain protein 1 | −3.48669 |
| 9938 | ARHGAP25 | Rho GTPase activating protein 25 | −3.56951 |
| 51669 | TMEM66 | transmembrane protein 66 | −3.74104 |
| 51101 | C8orf70 | chromosome 8 open reading frame 70 | −3.78615 |

(**Weight = gene-specific coefficient or reference value)

### REFERENCES

1. Jemal A, Siegel R, Ward E, Murray T, Xu J, Thun M J. Cancer Statistics, 2007. CA Cancer J Clin 2007:57:43-66.
2. Arriagada R, Bergman B, Dunant A, Le Chevalier T, Pignon J P, Vansteenkiste J. Cisplatin-based adjuvant chemotherapy in patients with completely resected non-small-cell lung cancer, N Engl J Med 2004:350:351-60.
3. Winton T, Livingston R, Johnson D, et al. Vinarelbine plus cisplatin vs. observation in resected non-small-cell lung cancer. N Engl J Med 2005; 352:2589-97.
4. Douillard J Y, Rosell R, De Lena M, et al. Adjuvant vinorelbine plus cisplatin versus observation in patients with completely resected stage I B-WA non-small-cell lung cancer (Adjuvant Navelbine International Trialist Association [ANITA]): a randomised controlled trial. Lancet Oncol 2006; 7:719-27.
5. Strauss G M, Herndon J E, II, Maddaus M A, et al. Adjuvant chemotherapy in stage IB non-small cell lung cancer (NSCLC): Update of Cancer and Leukemia Group B (CALGB) protocol 9633. ASCO Meeting Abstracts 2006; 24:7007-.
6. Pignon J P, Tribodet H, Scagliotti G V, et al. Lung Adjuvant Cisplatin Evaluation (LACE): A pooled analysis of five randomized clinical trials including 4,584 patients. ASCO Meeting Abstracts 2006; 24:7008-,
7. Scagliotti G V, Fossati R, Torri V. et al. Randomized study of adjuvant chemotherapy for completely resected stage I, II, or WA non-small-cell Lung cancer. J Natl Cancer Inst 2003; 95:1453-61.
8. Waller D, Peake M D, Stephens R J, et al. Chemotherapy for patients with non-small cell lung cancer: the surgical setting of the Big Lung Trial, Eur Cardiothorac Surg 2004; 26:173-82.
9. Douillard J Y, Rosell R, Delena M, Legrourmellec A. Torres A, Carpagnano F. ANITA: Phase III adjuvant vinorelbine (N) and cisplatin (P) versus observation (OBS) in completely resected (stage I-III) non-small-cell lung cancer (NSCLC) patients (pts): Final results after 70-month median follow-up. On behalf of the Adjuvant Navelbine International Trialist Association. ASCO Meeting Abstracts 2005; 23:7013-.
10. Hoffman P C, Mauer A M, Vokes E E. Lung cancer. Lancet 2000; 355:479-85.
11. Nesbitt J C, Putnam J B, Jr., Walsh G L, Roth J A, Mountain C F. Survival in early-stage non-small cell lung cancer. Ann Thorac Surg 1995; 60:466-72,
12. Beer D G, Kardia S L, Huang C C, et al. Gene-expression profiles predict survival of patients with lung adenocarcinoma. Nat Med 2002; 8:816-24.
13. Chen H Y, Yu S L, Chen C H, et al. A five-gene signature and clinical outcome in non-small-cell lung cancer. N Engl J Med 2007; 356:11-20,
14. Lu Y, Lemon W, Liu P Y, et al. A gene expression signature predicts survival of patients with stage non-small cell lung cancer. PLoS Med 2006; 3:e467,
15. Potti A, Mukherjee S, Petersen R, et al. A genomic strategy to refine prognosis in early-stage non-small-cell lung cancer. N Engl J Med 2006; 355:570-80.
16. Raponi M, Zhang Y, Yu J, et al. Gene expression signatures for predicting prognosis of squamous cell and adenocarcinomas of the lung. Cancer Res 2006; 66:7466-72.
17, Wigle D A, Jurisica I, Radulovich N, et al. Molecular profiling of non-small cell lung cancer and correlation with disease-free survival. Cancer Res 2002; 62:3005-8.
18. Bianchi F, Nuciforo P, Vecchi M, et al. Survival prediction of stage lung adenocarcinomas by expression of 10 genes, J Clin Invest 2007; 117:3436-44.
19. Sun Z, Wigle D A, Yang P. Non-overlapping and non-cell-type-specific gene expression signatures predict lung cancer survival, J Clin Oncol 2008; 26:877-83.
20. Lau S K, Boutros P C, Pintilie M, et al. Three-gene prognostic classifier for early-stage non small-cell lung cancer. J Clin Oncol 2007; 25:5562-9.
21. Tibshirani, R. 1997. The lasso method for variable selection in the Cox model. Statistics in Medicine 1997, 16:385-395.
22. R Development Core Team (2010). R: A language and environment for statistical computing, reference index version 2.10.1. R Foundation for Statistical Computing, Vienna, Austria. ISBN 3-900051-07-0, URL http://www.R-project.org.

1. A method for predicting survival of a patient with non-small cell lung cancer (NSCLC), the method comprising:
determining a gene expression profile from a sample of the patient's lung tumor, the gene expression profile comprising the level of expression of at least 5 genes from Table 3, and
classifying the gene expression profile as being predictive of good survival or poor survival.

2. The method of claim 1, wherein the gene expression profile further comprises the level of expression of one or more normalization genes in the sample.

3. The method of claim 1, wherein the gene expression profile is determined by hybridization-based assay or quantitative PCR.

4. The method of claim 1, wherein the sample is a frozen tissue specimen, or is a formalin-fixed paraffin-embedded tumor tissue sample, or is a cultured tumor specimen.

5. A method for preparing a gene expression profile indicative of response to adjuvant chemotherapy for non-small cell lung cancer (NSCLC), comprising:
determining the level of expression of at least 5 genes from Table 3 from a tumor tissue sample from a NSCLC patient.

6. The method of claim 5, wherein the expression levels for at least 10 genes from Table 3 are determined.

7. The method of claim 5, wherein the expression levels of fewer than about 1000 genes are determined.

8. A method of prognosing or classifying a subject with non-small cell lung cancer (NSCLC) comprising the steps:
a. determining the expression of biomarkers in a test sample from the subject, wherein the biomarkers correspond to at least 5 genes in Table 3, and
b. comparing the expression of the biomarkers in the test sample with expression of the biomarkers in a control sample,
wherein a difference or a similarity in the expression of the forty biomarkers between the control and the test sample is used to prognose or classify the subject with NSCLC into a poor survival group or a good survival group.

9. The method of claim 8, wherein the biomarker reference expression profile comprises a poor survival group or a good survival group.

10. The method of claim 8 wherein the NSCLC is stage I B or stage II.

11. The method of claim 8, wherein the expression level of the biomarkers is measured by a technique selected from the group consisting of quantitative PCR, nanostring technology, or microarray analysis.

12. The method of claim 11, wherein the expression level of the biomarkers is measured by microarray analysis.

13. The method of claim 12, comprising measuring expression level with a U133A chip.

14. The method of claim 11, wherein the biomarker expression levels is determined by quantitative PCR using Locked Nucleic Acid probes.

15. (canceled)

16. (canceled)

17. (canceled)

18. A composition comprising a plurality of isolated nucleic acid sequences, wherein each isolated nucleic acid sequence hybridizes to:
a. an RNA product of one of the 40 genes listed in Table 3; and/or
b. a nucleic acid complementary to a),
wherein the composition is used to measure the level of RNA expression of the 40 genes or subset thereof.

19. An array comprising at least one polynucleotide probe hybridizable to an expression product of each of the 40 the gene listed in Table 3, or subset thereof of at least 5 genes listed in Table 3, wherein the array contains fewer than 2000 probes.

20. (canceled)

21. A computer implemented product for predicting a prognosis or classifying a subject with NSCLC comprising:
a. a means for receiving values corresponding to a subject expression profile in a subject sample; and
b. a database comprising a reference expression profile associated with a prognosis, wherein the subject biomarker expression profile and the biomarker reference profile each has forty values, each value representing the expression level of a biomarker, wherein each biomarker corresponds to one gene in Table 3;
wherein the computer implemented product selects the biomarker reference expression profile most similar to the subject biomarker expression profile, to thereby predict a prognosis or classify the subject.

22. (canceled)

23. (canceled)

24. (canceled)

25. A computer system comprising
a. a database including records comprising a biomarker reference expression profile of forty genes in Table 3 associated with a prognosis or therapy;
b. a user interface capable of receiving a selection of gene expression levels of the 40 genes in Table 3 for use in comparing to the biomarker reference expression profile in the database;
c. an output that displays a prediction of prognosis or therapy according to the biomarker reference expression profile most similar to the expression levels of the forty genes.

26. A kit to prognose or classify a subject with early stage NSCLC, comprising detection agents that can detect the expression products of 40 biomarkers or subset thereof, wherein the biomarkers comprise at least 5 genes in Table 3.

27. A method of prognosing or classifying a subject with non-small cell lung cancer comprising:
a. determining the relative expression of at least 5 biomarkers in a test sample from the subject, wherein the biomarkers correspond to genes in Table 3,
b. multiplying the relative expression of each of the biomarkers by a reference value for the corresponding biomarker,
c. calculating a risk score for the test sample by summing the values obtained in step (b), and
d. comparing the risk score calculated for the test sample with a control value,
wherein a risk score above said control value is used to prognose or classify the subject with NSCLC into a good survival group and a risk score below said control value is used to prognose or classify the subject with NSCLC into a poor survival group.

28. A method of prognosing a subject with NSCLC comprising:
(a) determining relative expression levels of at least 5 biomarkers from Table 3,
(b) calculating a risk score for the subject from the expression levels of said biomarkers, and,
(c) comparing the risk score to a control value,
wherein a risk score greater than the control value is used to classify a subject into a high risk or poor survival group and a risk score lower than the control value is used to classify a subject into a lower risk or good survival group.

29. The method according to claim 20, wherein the at least 40 biomarkers comprise AK3L1, MAZ, MYH7B, MYCN, WDR4, MBDS, ARD1A, ZNF343, INHBE, WDR39, TACR2, LOC441601, SLC16A1, SLC27AS, KCNJ12, POLD3, RPA3, NEUROG1, LSMS, IGSF6, TRAK2, RAFTLIN, TRAF3IP3, CTA-246H3.1, TNFRSF17, IGHG1, RAPGEF2, P2RY10, CFLAR, RAI17, MTUS1, CNGB1, PPM2C, EBAG9, BTK, SRP9, EPAS1, ARHGAP25, TMEM66, and C8orf70

30. The method of claim 27, wherein the NSCLC stage is selected from the group consisting of stage I-A, stage I-B, or stage II.

31. The method of claim 27, wherein the risk score is the sum, across the biomarkers, of the inner product of a reference value and the relative expression level for each biomarker.

32-39. (canceled)
